(19) United States
(12) Patent Application Publication
(10) Pub. No.: US 2025/0204898 A1
(43) Pub. Date: Jun. 26, 2025

(54) DEVICES AND METHODS FOR FACILITATING THE ADVANCEMENT OF TREATMENT DEVICES FOR NEUROVASCULAR PROCEDURES

(71) Applicant: Baylis Medical Technologies Inc., Mississauga (CA)

(72) Inventors: Amanda Hartley, Caledon (CA); Luke Keaveney, Georgetown (CA); Amanda Centazzo-Colella, Bowmanville (CA); Ahmad Khayer Dastjerdi, Toronto (CA); Patrick Ryan, Toronto (CA); Jacob Eitan Miller, Toronto (CA)

(21) Appl. No.: 18/982,131

(22) Filed: Dec. 16, 2024

Related U.S. Application Data
(60) Provisional application No. 63/613,828, filed on Dec. 22, 2023.

(51) Int. Cl. A61B 17/00; A61B 17/12; A61B 17/22

(52) U.S. Cl. CPC A61B 17/00234; A61B 17/12113; A61B 17/22; A61B 2017/00238; A61B 2017/00305; A61B 2017/00367; A61B 2017/00557; A61B 2017/00778; A61B 2017/1205; A61B 2017/22055; A61B 2017/22069

(57) ABSTRACT

Systems and methods are disclosed for facilitating the advancement of treatment devices within the neurovascular system of a patient. The method includes advancing an outer device, while in a manoeuvrable configuration, to a first location and actively bracing the outer device in a braced configuration, such that the position of the outer device is substantially maintained while one or more inner devices or treatment devices are advanced further into the neurovascular system. Active bracing features limit longitudinal movement of the devices, thereby reducing the risk of one or more devices slipping or prolapsing out of a target blood vessel within tortuous anatomy.

### TECHNICAL FIELD

The disclosure relates to medical devices, and more particularly to methods and devices for facilitating advancement of treatment devices through tortuous anatomy for neurological applications.

### BACKGROUND OF THE ART

Neurological procedures require advancing medical devices through tortuous anatomy. For certain procedures it is imperative to complete the operation as fast as possible. For example, for patients suffering from acute ischemic stroke, a mechanical thrombectomy is performed to remove an embolism from the artery restoring blood flow to the brain.

Typical treatment methods include gaining femoral access and advancing a catheter access system (guidewire, diagnostic catheter and guide sheath) to the aortic arch, then the cannulating one of the three aortic branches that lead to the head, for example, the brachiocephalic artery. Next, the catheter access system is advanced further into the carotid artery until the access catheter is stable in the neurovascular system, which allows a conduit for introduction of thrombectomy devices. The guidewire and diagnostic catheter are removed and thrombectomy treatment devices (stent retriever, aspiration catheter, microcatheter and microwire/micro-guidewire) are introduced to either aspirate or withdraw the thrombus and recanalize the artery.

Certain anatomies pose greater challenges than others, including type III aortic arches, bovine arches and tortuous vessels. This anatomy creates challenges for advancing and stabilizing the catheter access system in the neurovascular system. In some instances, when a physician attempts to advance the access catheter into the aortic branch, the access catheter slips and prolapses out into the aorta. The access catheter must be stabilized in the target anatomical location in order for a physician to introduce mechanical thrombectomy devices to recanalize the affected vasculature.

Unstable access can also cause the guide sheath and/or diagnostic catheter to dislodge at other points in the procedure, including during therapy delivery. Securing the access catheter in a timely manner is crucial as mechanical thrombectomy efficacy is reduced with prolonged procedure time. Increased procedure time means decrease in the reversibly damaged area of brain tissue for the patient.

In addition to the device(s) falling back when trying to catheterize the carotid artery, one or more devices can also slip when advancing the access catheter and/or guide sheath distally into the neurovasculature, such as when the thrombectomy devices are introduced, or when the clot is retracted/aspirated. It is therefore advantageous for the access catheter to have features that prevent slipping or prolapsing into aorta throughout the entire procedure.

### BRIEF DESCRIPTION OF THE DRAWINGS

In order that the invention may be readily understood, embodiments of the invention are illustrated by way of examples in the accompanying drawings, in which:

FIG. 1A is an illustration of the anatomy of the aortic arch and surrounding blood vessels;

FIGS. 1B to 1D are illustrations of various types of aortic arches;

FIG. 2 is an illustration of a catheter access and delivery system for facilitating the treatment of neurological procedures in accordance with an embodiment of the present invention;

FIGS. 3A to 3C are illustrations showing the challenges encountered when advancing devices within tortuous blood vessels;

FIGS. 4A is an illustration of an articulating catheter in accordance with an embodiment of the present invention;

FIGS. 4B is an illustration of the distal end of the articulating catheter in accordance with an embodiment of the present invention;

FIG. 4C is a cross-section of an articulating catheter shaft in accordance with an embodiment of the present invention;

FIG. 4D is an illustration of an articulating catheter in accordance with an embodiment of the present invention;

FIG. 5 is an illustration of an articulating catheter adopting a curved configuration in the aortic arch in accordance with an embodiment of the present invention;

FIG. 6 is an illustration of a plurality of devices adopting a curved configuration in the aortic arch in accordance with an alternative embodiment of the present invention;

FIG. 7A is an illustration of an articulating catheter actively bracing against a vessel wall in accordance with an embodiment of the present invention;

FIG. 7B is an illustration of an articulating catheter actively bracing against a vessel wall in accordance with an alternative embodiment of the present invention;

FIGS. 8A and 8B are illustrations of an access catheter comprising a non-occlusive balloon assembly in accordance with an embodiment of the present invention;

FIG. 8C is an illustration of a non-occlusive balloon assembly in accordance with an alternative embodiment of the present invention;

FIGS. 8D and 8E are illustrations of a non-occlusive balloon assembly in accordance with a further embodiment of the present invention;

FIGS. 9A and 9B are illustrations of a sheath-catheter assembly comprising an expandable anchoring mechanism in accordance with an embodiment of the present invention;

FIG. 9C is an illustration of an access catheter comprising an outer sheath and an expandable scaffold in accordance with an embodiment of the present invention;

FIG. 9D is an illustration of an access catheter comprising a ring scaffold in accordance with an alternative embodiment of the present invention;

FIG. 10 is a flow diagram showing a method of advancing devices within blood vessels as part of a procedure for treating a neurological condition in accordance with an embodiment of the present invention;

FIGS. 11A to 11F illustrate a method of facilitating advancement of devices within a neurovascular system in accordance with an embodiment of the present invention;

FIG. 12 is a flow diagram showing a method of advancing devices within blood vessels as part of a procedure for treating a neurological condition in accordance with an alternative embodiment of the present invention;

FIGS. 13A to 13E illustrate a method of facilitating advancement of devices within a neurovascular system in accordance with an alternative embodiment of the present invention; and

FIGS. 14A and 14B are illustrations of an elongate device capable of bracing against vessel walls in accordance with a further embodiment of the present invention.

In the drawings, like reference characters generally refer to the same parts throughout the different views. Also, the drawings are not necessarily to scale, emphasis instead being placed upon generally illustrating the various concepts discussed herein.

### DETAILED DESCRIPTION

Human neurovascular anatomy can be difficult to navigate because of its tortuous nature. There are numerous neurovascular procedures that require advancing treatment devices though this anatomy. Some examples of such procedures include the following: the treatment of acute ischemic stroke, endovascular coil embolization, treating arteriovenous malformations (AVMs), a cerebral venous sinus thrombosis procedure, treating chronic subdural hematoma, and carotid angioplasty and stenting.

The systems and methods for facilitating advancement of treatment devices described herein may be used for, but are not limited to, any of the procedures described herein. Various solutions described herein will be illustrated with respect to a mechanical thrombectomy procedure for treatment of acute ischemic stroke, but other applications are possible, and examples should not be considered limiting.

In one broad aspect, embodiments of the present invention comprise a method for positioning a device within a target blood vessel, the method using a medical device assembly including an inner device and an outer device, the outer device configured to have a maneuverable configuration for advancing through vasculature, and a braced configuration for supporting advancement of the inner device therethrough, the method comprising the steps of: advancing the outer device to a first location within a patient's body while the outer device is in the maneuverable configuration; causing the outer device to adopt the braced configuration at the first location; and advancing the inner device through the outer device into the target blood vessel while the outer device is maintained in the braced configuration; and while in the braced configuration, the outer device is configured to substantially maintain its position at the first location to facilitate advancement of the inner device therethrough to the target blood vessel.

As a feature of this aspect while in the braced configuration, the outer device comprises a curved orientation.

As a feature of this aspect, the curved orientation comprises an S-shaped curve.

As a feature of this aspect, the first location is within the target blood vessel, and a curved portion of the outer device is in contact with a wall of the target blood vessel while in the braced configuration.

As a feature of this aspect, the first location is within the target blood vessel, and a first curved portion of the outer device and a second curved portion of the outer device are in contact with a wall of the target blood vessel while in the braced configuration.

As a feature of this aspect, the step of causing the outer device to adopt the braced configuration comprises deploying an anchoring mechanism.

As a feature of this aspect, the anchoring mechanism comprises a non-occlusive balloon assembly.

As a feature of this aspect, the non-occlusive balloon assembly comprises a plurality of balloons.

As a feature of this aspect, the anchoring mechanism comprises an expandable element.

As a feature of this aspect, the expandable element comprises a scaffold.

As a feature of this aspect, the method further comprises a step of locking the outer device in the braced configuration.

As a feature of this aspect, the step of locking the outer device comprises manipulating a locking mechanism.

As a feature of this aspect, the method further comprises a step of, following the step of advancing the inner device, causing the outer device to adopt the maneuverable configuration and advancing the outer device to a second location, and the second location is closer to the target blood vessel than the first location.

As a feature of this aspect, the method further comprises a step of, following the step of advancing the inner device, causing the outer device to adopt the maneuverable configuration and advancing the outer device into the target blood vessel.

As a feature of this aspect, the target blood vessel is selected from the group consisting of a right common carotid artery, a left common carotid artery, an internal common carotid artery, a left subclavian artery, a right subclavian artery, and a brachiocephalic artery.

As a feature of this aspect, advancing the outer device to the first location includes advancing the outer device within an aorta.

As a feature of this aspect, the aorta comprises a Type III aortic arch.

As a feature of this aspect, the method further comprises a step of advancing at least one treatment device through the outer device.

As a feature of this aspect, the method further comprises a step of treating a neurological condition using the at least one treatment device.

As a feature of this aspect, treating the neurological condition comprises removing a clot.

As a feature of this aspect, treating the neurological condition comprises widening narrowed carotid arteries and deploying a stent.

As a feature of this aspect, treating the neurological condition comprises occluding an aneurysm within a cerebral vessel.

As a feature of this aspect, treating the neurological condition includes alleviating intracranial pressure.

As a feature of this aspect, the step of causing the outer device to adopt a braced configuration comprises manipulating an actuator.

As a feature of this aspect, the step of causing the outer device to adopt a braced configuration comprises manipulating an actuator, and the actuator is coupled to a first location and a second location on the outer device.

As a feature of this aspect, the step of causing the outer device to adopt a braced configuration comprises manipulating a first actuator and a second actuator, the first actuator coupled to a first location on the outer device and the second actuator coupled to a second location on the outer device.

As a feature of this aspect, causing the outer device to adopt the braced configuration includes aiming a distal end of the outer device towards the target blood vessel.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

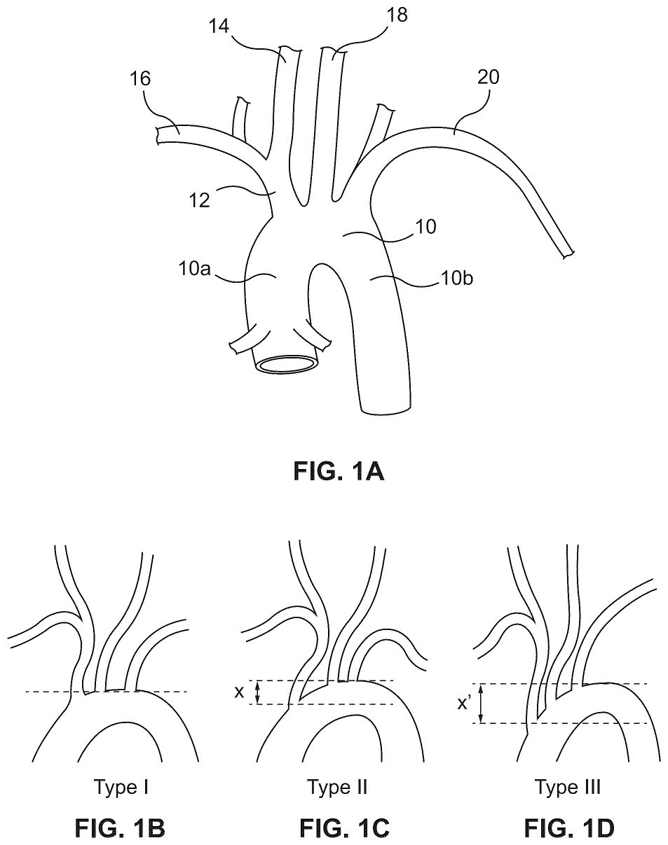

FIG. 1A is a diagram of neurovasculature within a patient's body showing aortic arch 10 including ascending aorta 10a and descending aorta 10b, left common carotid artery 18, left subclavian artery 20, and brachiocephalic artery 12 leading to the right common carotid artery 14 and right subclavian artery 16.

FIGS. 1B to 1D show aortic arch classification where: Type I is normal, Type II being a descended arch, and Type III being an extremely descended arch. For a Type II, the arch has descended a distance X compared to Type I, and for Type III, the arch has descended a distance X′, where X′ is greater than X. Type III is the most tortuous and most clinically challenging case. The solutions described herein will be described with reference to a Type III arch but are applicable to all three types.

### Catheter Access System

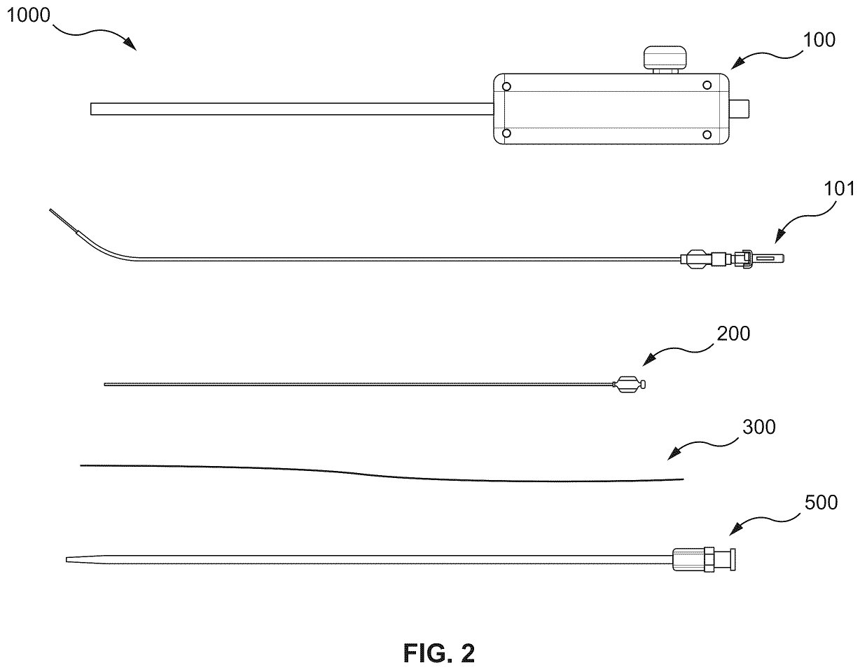

With reference to FIG. 2, a catheter access system is shown that includes a plurality of medical devices which can be used to facilitate advancement of one or more treatment devices for a neurovascular procedure described herein. According to one embodiment or the present invention, catheter access system 1000 includes an articulating catheter 100, a diagnostic catheter 200, and a guidewire 300.

In an alternative embodiment, catheter access system 1000 includes a non-steerable access catheter 101, a diagnostic catheter 200, and a guidewire 300. Access catheter 101 does not have active steering features, i.e., it does not comprise any actuators to manipulate that deflect a portion of the shaft. Access catheter 101 is a flexible catheter with a similar construction to known access catheters or guide sheaths and further comprises at least one anchoring feature described herein. In some embodiments, access catheter 101 has a pre-curved distal end.

Any embodiment described herein is configured to receive “treatment devices” which can be used as part of an overall procedure to treat a neurological condition. Treatment devices can include, but are not limited to, a stent retriever, an aspiration catheter, a microwire, a guide wire, a microcatheter, an intermediate catheter, a distal access catheter, a wire, or a device for delivering an embolic agent.

In some embodiments, catheter access system 1000 includes a dilator 500, which may be used at the beginning of the procedure to facilitate delivering the components of the catheter access system 1000 into the patient.

In one embodiment, guidewire 300 may be any guidewire or wire guide, known by those skilled in the art, for example a flexible wire having a 0.035″ or 0.038″ outer diameter.

In one embodiment, diagnostic catheter 200 may be similar to a know known flexible catheter, for example, the Penumbra Select™ diagnostic catheter or any of the Beacon® catheters by Cook Medical (SIM, VTK, JB, etc.). In some embodiments, diagnostic catheter 200 comprises a pre-curved/fixed curve distal region (not shown).

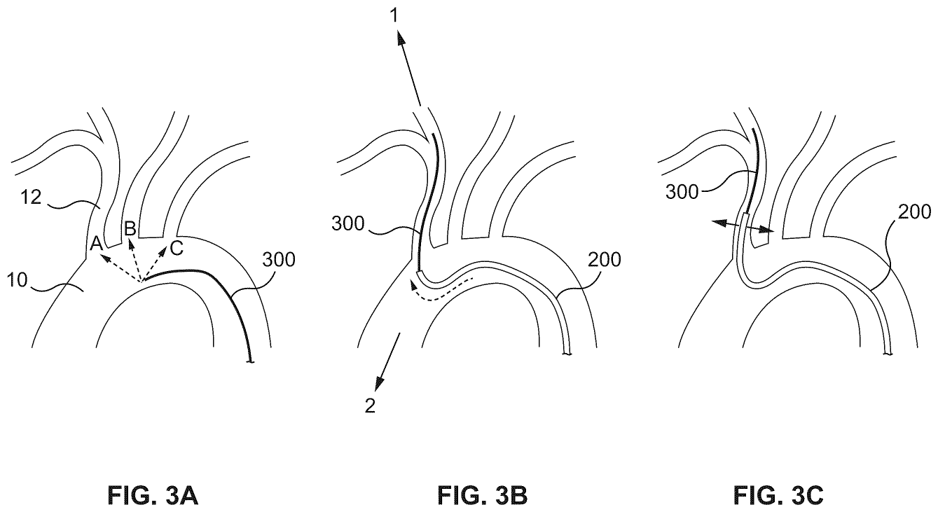

FIGS. 3A to 3C illustrate how multiple devices function together in tortuous anatomy and the challenges faced with neurological procedures, for example a mechanical thrombectomy for treatment of acute ischemic stroke. FIG. 3A shows a guidewire 300 located in the aortic arch 10. For an acute ischemic stroke, an occlusion may be located somewhere in the internal carotid artery or middle cerebral artery. As a first step in the procedure, the guidewire 300 must access the correct blood vessel so that it can ultimately reach the carotid artery. Three possible choices of blood vessels are shown by dashed arrows A, B, C, and in one example, the desired blood vessel is arrow A, while avoiding arrows B or C. In certain instances, for example Type III aortic arches, it can be challenging to select the appropriate blood vessel due to the curvature of anatomy.

A diagnostic catheter 200 may be used to aim a wire guide towards a target blood vessel, for example the brachiocephalic artery 12. Once the guidewire 300 has advanced in the right common carotid artery, shown in FIG. 3B, diagnostic catheter 200 (and/or access catheter 101) needs to be advanced deeper into the right common carotid artery, over the guidewire, shown as arrow 1. Because of the tortuous nature of the Type III arch, the diagnostic catheter 200 exerts a force, shown by arrow 2, that tends to force the diagnostic catheter 200 and guidewire 300 to prolapse out of the target vessel and into the ascending aorta 10a. In other words, there is a force pushing the devices “downwards”. This force arises from the characteristics of the diagnostic catheter 200/access catheter 101, as they have a tendency to straighten.

In known procedures, once the devices are advanced far enough into the carotid artery, diagnostic catheter 200 passively braces against the vessel wall, as shown by lateral arrows in FIG. 3C. Passively bracing means the device braces against the wall due to its natural tendency to bend and exert some radial force on the vessel wall. At later steps in the procedure, even with passive bracing, as other devices are removed or introduced, one or all devices may slip or move downwards, or even prolapse due to the downward forces described above.

The inventors have developed novel solutions to 1) aid in accessing a targeted blood vessel and 2) add additional bracing/stabilizing features to the access catheter, including using the vessel walls, to reduce the risk of prolapse. These additional bracing features are “active” bracing features. In other words, a physician can manipulate some feature (actuator) on the articulating catheter 100 proximal end, such that the catheter switches from a first, flexible configuration, to a second, braced or biased configuration. The flexible (maneuverable) configuration is used when advancing a device within a blood vessel, and the braced configuration is used to maintain the position of the catheter while inner devices are moved therethrough and resist the tendency to prolapse. In other words, to anchor the device in place. In some examples, bracing, stabilizing, or anchoring may be used interchangeably.

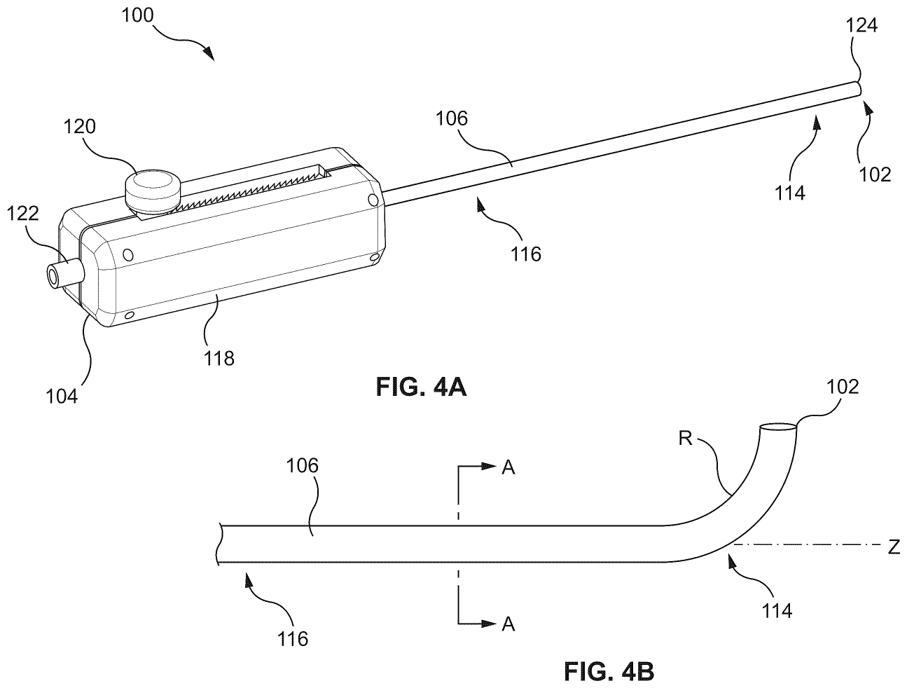

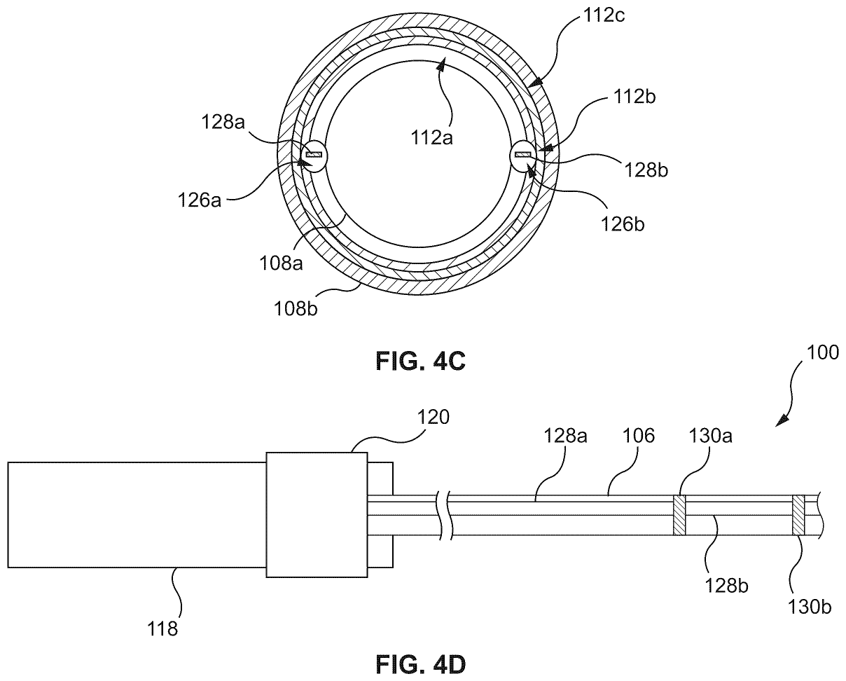

Referring to FIGS. 4A to 4D, according to one embodiment of the present invention, articulating catheter 100 has a distal end 102, a proximal end 104, and includes a shaft 106 comprising inner wall 108a and outer wall 108b defining a primary lumen 110 which terminates at distal opening 124.

Articulating catheter 100 further comprises a handle 118 located at the proximal end 104, the proximal end of shaft 106 being coupled to the handle 118. Shaft 106 extends through handle 118 and is fluidly coupled to hub 122. Handle 118 comprises one or more actuators 120, and a hub 122. Actuator 120 may be any commonly known device such as a knob, wheel, lever, or slider. In some embodiments, hub 122 may be a luer connection, a (rotating) hemostatic valve, or other known mechanism used to receive an inner device, for example, diagnostic catheter 200 or guidewire 300, through primary lumen 110 such that the inner device can exit through distal opening 124. Hub 122 and primary lumen 110 are also configured to receive one or more treatment devices.

In one embodiment, shaft 106 comprises a distal region 114 and a proximal region 116, where distal region 114 is more flexible than proximal region 116, such that it can more easily deflect away from a device longitudinal axis Z, as shown in FIG. 4B. Distal region 114 has a bend radius R. In some embodiments, shaft 106 comprises one or more regions of varying stiffness to achieve various curved configurations. Regions of varying stiffness may be achieved using materials having different stiffness properties.

FIG. 4C shows a cross section of shaft 106 taken along line A-A in FIG. 4B. In one embodiment, shaft 106 comprises one or more layers 112, for example a first layer 112a, second layer 112b, and third layer 112c. In one example, first layer 112a comprises a lubricious polytetrafluorethylene “PTFE” (or similar material), second layer 112b comprises a reinforced layer such as a braid, coil, or laser cut hypotube, and third layer 112c comprises a reflown thermoplastic layer. Shaft 106 also defines one or more minor lumens 126 located between inner wall 108a and outer wall 108b. In some embodiments, minor lumens 126 are located under second layer 112b, in other embodiments, minor lumens 126 are above second layer 112b.

In one embodiment, articulating catheter 100 further comprises one or more pull wires 128 located within the one or more minor lumens 126. At the proximal end of the device, pull wires 128 are connected to one or more actuators 120 located in handle 118. The distal end of each pull wire 128 is coupled to one or more pull rings 130, embedded within shaft 106, in the distal region 114. In one example, pull wires 128 are laser welded to pull rings 130.

As is known in steerable sheath device construction and operation, when the actuator is actuated, pull wire 128 is tensioned and distal region 114 deflects or curves. In other words, it adopts a curved configuration. The curved configuration changes the profile of shaft 106, and one feature of this allows the device to be “steered” or “aimed” within the vasculature. In other words, “aiming” means positioning the distal end so that the distal opening 124 facing a desired location, for example pointing at a target blood vessel, and any device that exits the distal opening will advance toward the target location.

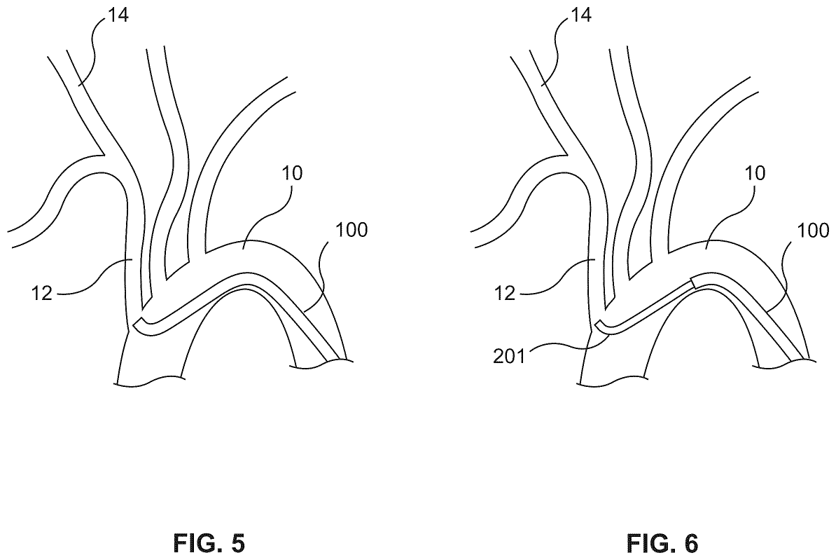

In one embodiment, articulating catheter 100 comprises two pull wires 128a, 128b, each connected to a separate pull ring 130a, 130b respectively, as shown in FIG. 4D. Pull wire 128b passes over, under, or through a channel located in pull ring 130a and is not physically connected to pull ring 130a. Such an embodiment allows the shaft 106 to adopt a double curve or “S-shaped” curve, shown in FIG. 5. Double curve or s-shaped curved means a first curve in a distal region and second curve proximal to the first curve, the second curve in a generally opposite direction to the first curve. In some embodiments, the radius of the first curve is different than the radius of the second curve. In some embodiments, pull wires 128a and 128b are radially offset by approximately 180 degrees.

In one embodiment, articulating catheter 100 comprises two actuators, 120a, 120b, (not shown) each actuator configured to control tensions of a single pull wire 128. In another embodiment, two pull wires are connected to a single actuator 120 located in the handle.

In one embodiment, articulating catheter 100 comprises one pull wire 128 connected to one pull ring 130. In another embodiment, articulating catheter 100 is a bi-directional articulating catheter and comprises two pull wires 128a, 128b, to allow for bi-directional steering, connected to pull ring 130 (not shown). In such an embodiment, both pull wires may be controlled by a single actuator 120. In some embodiments for a bi-directional articulating catheter, as pull wire 128a is retracted, pull wire 128b is advanced, to allow steering in a first direction, then as pull wire 128a is advanced, pull wire 128b is retracted to allow steering in the other direction. In some examples, the first and second directions are opposite each other, in other words, the pull wires are 180 degrees apart.

In some embodiments, distal region 114 may have an enhanced flexibility, such that it easily bends and offers little rigidity or resistance to bending forces exerted upon it. In other words, it comprises a floppy distal region, which may add in navigation and vessel selection. In some embodiments, all of or portions of shaft 106 comprises a hydrophilic coating.

In further embodiments (not shown), articulating catheter 100 may comprise two pull rings, two actuators, and four pull wires running through four wire lumens, where each pull ring connected to one actuator via two pull wires. In still further embodiments, articulating catheter 100 has additional pull rings to allow multiple curved portions of the shaft, for example three or more curves.

In some embodiments, articulating catheter 100 comprises one or more locking features (not shown), in order to lock the curved configuration. Locking features may be located on the handle, and may use any of several known mechanisms, for example a switch, a push button, or a spring-loaded feature. Locking features may also comprise an auto-lock or self-locking feature, where some kind of action must be performed in order to adjust the deflection of shaft 106.

In some embodiments, articulating catheter 100 and/or access catheter 101 comprises radiopaque features that allow the device to be viewed under various known imaging modalities. Radiopaque features can include one or more marker bands, for example platinum iridium, located anywhere along shaft 106 (not shown). In other embodiments, one or more layers comprises a radiopaque filler that may provide visibility under fluoroscopy, for example, a concentration of BaSo4.

In some embodiments, articulating catheter 100 and/or access catheter 101 comprise one or more hypotubes. Hypotube may comprise 304-stainless steel, 316-stainless steel, nitinol, or any other suitable material. In one embodiment, hypotube comprises a laser-cut pattern configured to allow hypotube to deflect, for example a series of cutouts. In one specific example, hypotube defines a series of ‘C-shaped’ cutouts. In one embodiment, hypotube spans substantially the entire length of the shaft 106. In other embodiments, one or more hypotubes may be located proximal to one or more pull rings 130, and cutouts are configured to facilitate bending. One or more hypotubes may be used in combination with steering features described herein to facilitate the curved configurations of shaft 106.

One or more hypotubes may also be positioned at various locations along the shaft of access catheter 101 to support inner device advancement when strategically placed within the vasculature. For example, ‘C-shaped’ cutouts may allow access catheter 101 to deflect into the brachiocephalic artery 12, left common carotid artery 18, or left subclavian artery 20, and provide resistance to the downward forces exerted by the inner devices.

In another embodiment, a steerable catheter 201 may be used in place of diagnostic catheter 200. Steerable catheter 201 has articulating (or “steering”) features such that the distal end may be deflected or “steered” within the vasculature. Components on steerable catheter 201 are similar to those on articulating catheter 100 described herein. In such an embodiment, steerable catheter 201 comprises a handle on its proximal end connected to an elongate member or shaft. Handle further comprises an actuator and a hub. Hub is configured to receive a smaller device, for example, guidewire 300. Manipulating actuator will cause tension in a pull wire running the length of the shaft and connected to a pull ring, and cause the distal end of the shaft to deflect, thereby allowing steerable catheter 201 to be aimed within a blood vessel. Steerable catheter 201 may be used with either articulating catheter 100 or access catheter 101 and may assist in selecting an appropriate vessel.

In some embodiments, articulating catheter 100 and steerable catheter 201 may be used to aim the assembly using a double curve, or s-shaped curved, necessary to select the appropriate vessel, as shown in FIG. 6.

In other embodiments, a pre-curved diagnostic catheter 200 may be used with articulating catheter 100 to achieve the double curve shape. In other embodiments, a pre-curved diagnostic catheter 200 and a pre-curved access catheter 101 may be used to create a double curve (not shown).

### Bracing using curved configurations

Articulating/steerable features on the articulating catheter 100 may be used to both select a vessel and to provide active bracing for advancement of inner devices during a medical procedure, for example, a neurological procedure.

As described herein, when pull wires 128 are tensioned, a portion of the shaft 106 curves, or deflects away from a device longitudinal axis. In some embodiments, in the curved configuration, the curved portions of the shaft become more rigid. In some instances, curved (and tensioned) portions are rigid enough to resist the downward force exerted by inner devices as they are advanced through the tortuous anatomy. In other words, when the pull wire(s) is partially or fully tensioned, articulating catheter 100 adopts a braced configuration. When the wires are not tensioned, articulating catheter 100 has a maneuverable configuration that is flexible enough to traverse through vasculature more easily than in the braced configuration.

When positioned in the aortic arch 10 in a braced configuration, the articulating catheter 100 can aim towards the desired blood vessel, for example, brachiocephalic artery 12, shown in FIG. 5, and also resist downward forces described herein and shown in FIG. 3B (arrow 2). In one example, articulating catheter 100 comprises a double curve or s-shaped curve (two pull ring design shown in figure FIG. 4D) and is positioned in such a way where a proximal curve bends around the aortic arch 10 and a distal curve aims toward the brachiocephalic artery 12.

While the articulating catheter 100 is in this location within the vasculature and in the braced configuration, it is rigid enough to support advancement of the diagnostic catheter 200, or other device, further into the right common carotid artery 14 with reduced risk of prolapsing. The stiffness of the shaft 106 at the curves, (and in particular the distal curve) when in a braced configuration, resists the force exerted downward into the ascending aorta 10a. Therefore, the articulating catheter 100 substantially maintains its position within the aortic arch 10 as inner devices are advanced through primary lumen 110.

For clarity, “maintains its position” means limiting longitudinal movement such that the device effectively remains in the same position within a blood vessel, and allows an inner device to be advanced to the targeted vessel without the devices slipping or prolapsing. In other words, the devices do not prolapse or substantially move proximally such that they hinder the procedure. This method will be described in greater detail below.

Active bracing can also be achieved when articulating catheter 100 is located within a narrower vessel, for example the brachiocephalic artery 12 or right common carotid artery 14. The articulating catheter 100 can be advanced into a blood vessel while in the maneuverable configuration, and is then put into a braced configuration, such that at least one portion of shaft 106 deflects and comes into contact with a portion of the vessel wall. When an inner device is advanced through the articulating catheter, the force exerted by the inner device is partially absorbed by the vessel wall, stabilizing articulating catheter 100 and allowing it to maintain its longitudinal position within the vessel, and facilitate the advancement of the inner device without prolapsing. In other words, the articulating catheter 100 actively braces against the vessel wall to withstand the bending force imposed by the inner device.

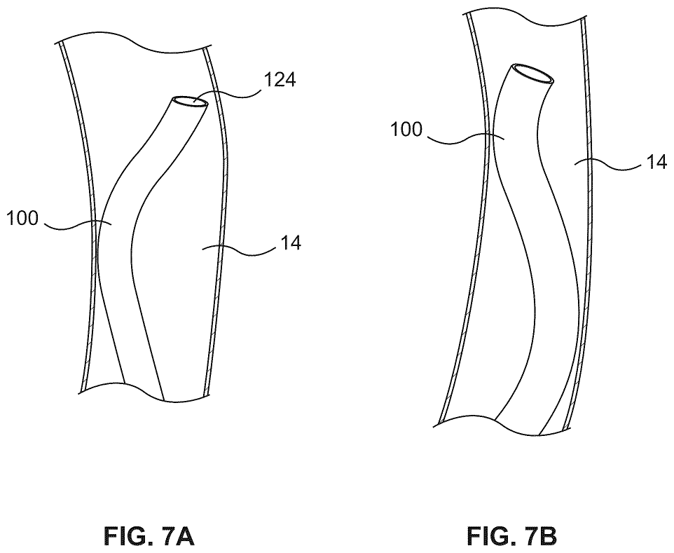

In one embodiment the articulating catheter 100 braced configuration comprises a single curve, shown in FIG. 7A. In another embodiment the braced configuration comprises a double curve, or s-shaped curve, as shown in FIG. 7B.

The radial force exhibited by the catheter on the vessel wall can act as an anchoring or “stabilizing” feature, such that the device is anchored to vessel wall and longitudinal movement of the device within the vessel is limited. In other words, the position of the catheter is maintained as inner devices are advanced or retracted.

### Non-occlusive Balloon Anchoring Mechanism

According to another embodiment of the present invention, in order to provide improved bracing capabilities compared with traditional access catheters, access catheter 101 comprises an anchoring feature or anchoring mechanism, located in a distal region. A method of using such features is described herein.

In some applications, balloons are used to arrest blood flow or reduce occlusions within blood vessels by compressing matter against a vessel wall. When these balloons are deployed/inflated they typically occlude the blood vessel and inhibit blood flow. In some applications, such as advancing devices prior to mechanical thrombectomy, it is desirable to keep blood flowing to the brain for at least part of the procedure.

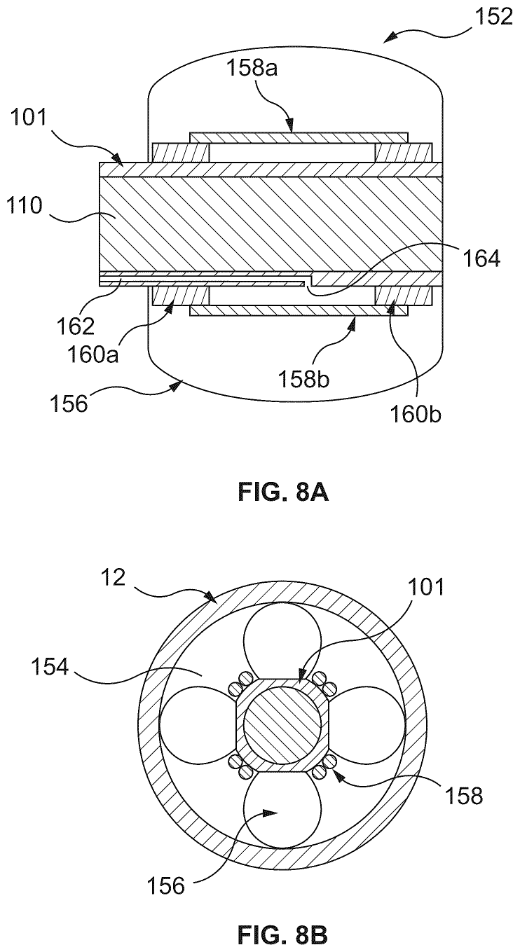

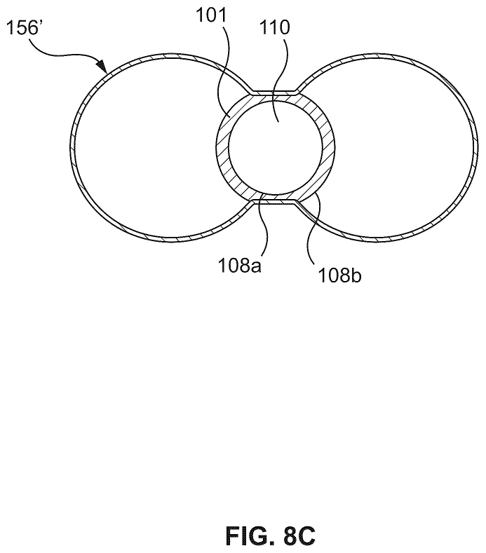

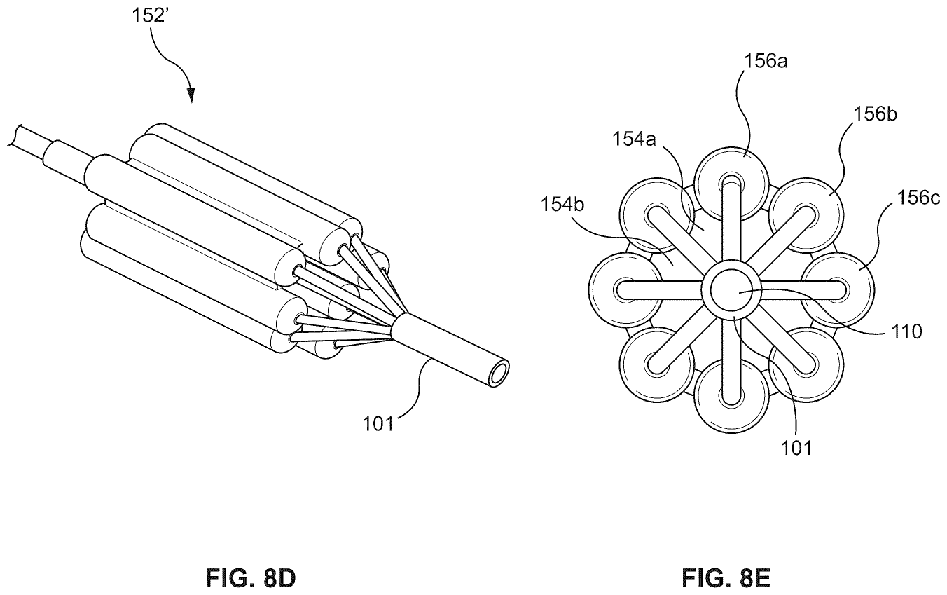

With reference to FIGS. 8A and 8B, in one embodiment, anchoring mechanism comprises a non-occlusive balloon assembly 152 that defines at least one channel 154, such that blood may flow through the vessel while the non-occlusive balloon assembly 152 is braced against a vessel wall. Non-occlusive balloon assembly 152 includes a balloon 156, and at least one restraining wire 158 affixed to the access catheter 101, configured such that when the balloon 156 is inflated, the restraining wire 158 compresses a segment of the balloon to define one or more channels 154. Balloon 156 is made of compliant or semi-compliant material. In some examples, balloon 156 comprises polyurethane, nylon, Pebax®, silicone, or Chronoprene®. Balloon 156 may be bonded to shaft 106 using reflow or heat shrink.

In one embodiment, non-occlusive balloon assembly 152 comprises a balloon 156 and a plurality of restraining wires 158, for example first wire 158a and second wire 158b. Each restraining wire 158 is affixed to first and second support structures 160a, 160b respectively, for example hypotubes, located on or within shaft 106. Shaft 106 further defines at least one inflation lumen 162, between inner wall 108a and outer wall 108b, and further defines an aperture 164 for inflating balloon 156 with a fluid.

Access catheter 101 proximal region (or handle 118) is configured to receive an inflation device (not shown) for inflating the balloon 156, by any known means. In one example, the device proximal end is connected to a bifurcated luer hub comprising a balloon inflation lumen and a through-lumen, which is configured to deliver inflation media to the balloon. When balloon 156 is inflated, portions of the balloon 156 come into contact with the vessel wall, (e.g., brachiocephalic artery 12), and other portions remain compressed by the restraining wires 158, as shown in FIG. 8B.

In another embodiment, shown in cross sectional view in FIG. 8C, balloon 156′ is configured such that it remains compressed against access catheter 101 outer wall 108b in at least one region, such that it when inflated, it takes an irregular shape that will not occlude a blood vessel, for example a figure-eight shape.

Referring to FIGS. 8D and 8E, in another embodiment, non-occlusive balloon assembly 152′ comprises a plurality of balloons, for example at least 152a, 152b, and 152c, etc., as shown in FIG. 8D. The plurality of balloons defines channels 154a, 154b, etc. to allow blood to flow. FIG. 8E shows a cross section of access catheter 101 and non-occlusive balloon assembly 152′. (NOTE not all ballons/channels are labeled in the figure)

### Expandable Scaffold Anchoring Mechanism

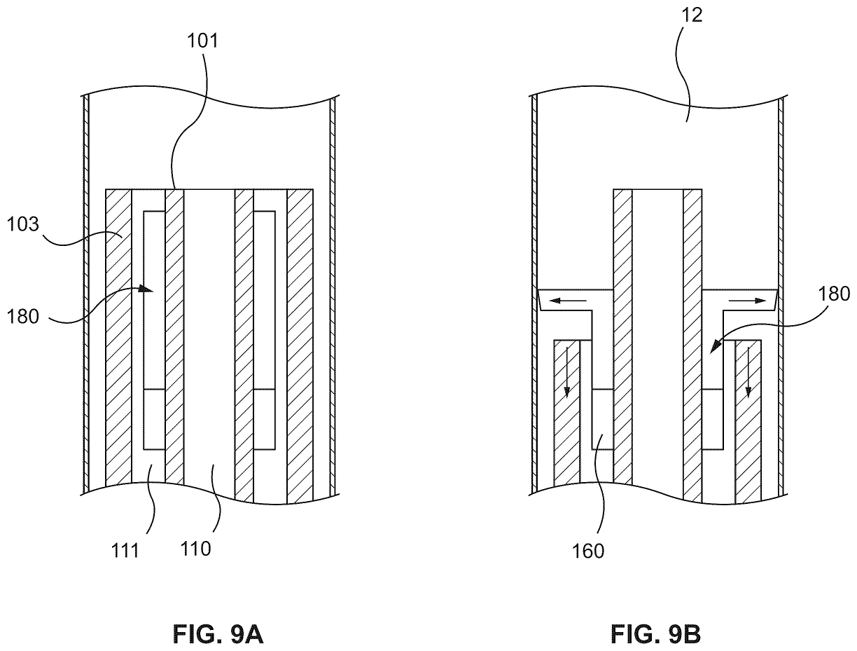

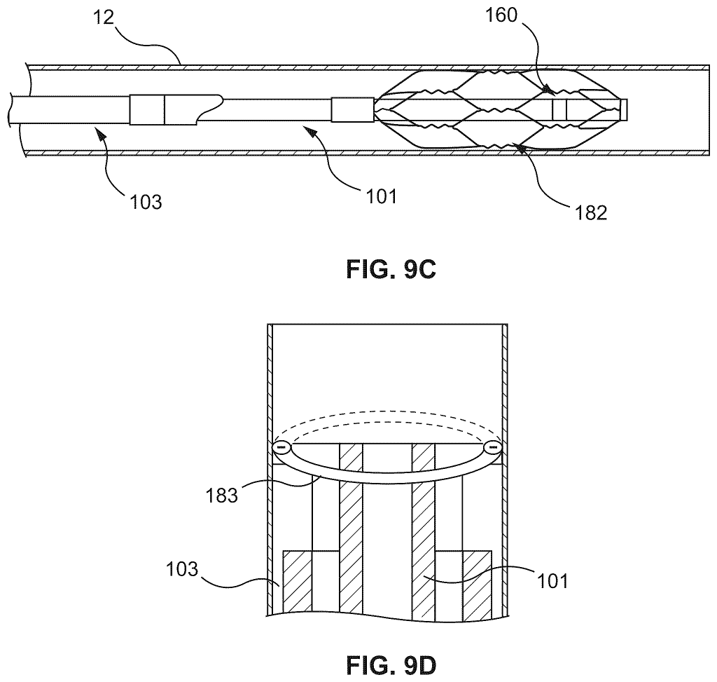

With reference to FIGS. 9A and 9B, in accordance with another embodiment or present invention, anchoring mechanism comprises an expandable element 180 affixed to the outer wall 108b of access catheter 101. Expandable element 180 may include any kind of compressible and self-expanding structure, having a variety of shapes and configurations. For example, expandable element 180 may be a filamentous element, wire coil, petals, a cage, or a spring-like structure. In some embodiments, expandable element 180 is affixed to a support structure 160, for example a hypotube, located on access catheter 101. In another embodiment, expandable element 180 is affixed to shaft 106 using reflow or heat-shrink.

In one embodiment, catheter access system 1000 additionally includes an outer sheath 103, defining a lumen 111 for receiving access catheter 101. Outer sheath 103 and access catheter 101 forming a sheath-catheter assembly. Outer sheath 103 is longitudinally moveable relative to access catheter 101 in order to deploy expandable element 180. Outer sheath 103 is sufficiently rigid, at least at the distal end, such that expandable element 180 remains in a compressed state when surrounded by outer sheath 103. In some embodiments, movement of outer sheath 103 may be controlled by a control mechanism, for example an actuator in the form of a slider, which may be part of a handle (not shown).

Sheath-catheter assembly has a first, compressed configuration, in which outer sheath 103 covers expandable element 180, and expandable element 180 is maintained in a compressed state/configuration, as shown in FIG. 9A. In one example, outer sheath 103 and access catheter 101 are distally aligned (distal ends at same position). When outer sheath 103 is retracted or access catheter 101 is advanced, sheath-catheter assembly switches to a second, expanded state/configuration where expandable element 180 expands and comes into contact with a vessel (e.g., brachiocephalic artery 12) wall, as shown in FIG. 9B.

In one specific example, expandable element 180 comprises a self-expanding scaffold 182. FIG. 9C shows scaffold 182 in an expanded state, braced against a brachiocephalic artery 12 vessel wall.

Referring to FIG. 9D, in another embodiment, expandable element 180 comprises a ring scaffold 183.

In some embodiments, access catheter 101 and/or outer sheath 103 include(s) one or more locking features (not shown) located at the proximal ends, to secure (or couple) outer sheath 103 and access catheter 101 such that they may be advanced or retracted as a single device. Locking features may be decoupled to allow the access catheter 101 and outer sheath 103 to move with respect to each other, for example when deploying the expandable element 180.

### Expandable Body Comprising Slots

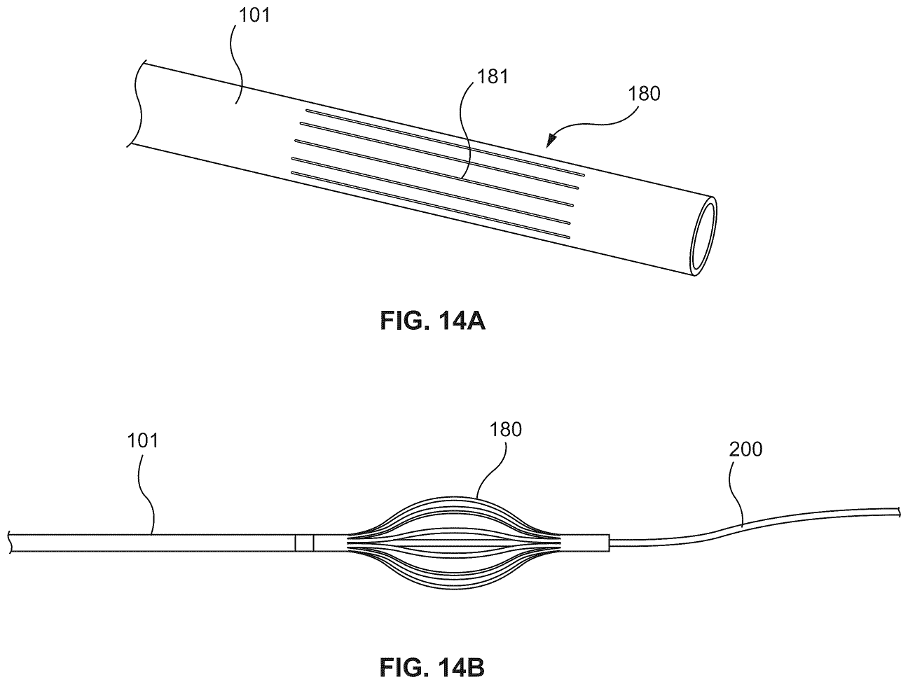

With reference to FIGS. 14A, and 14B, in another embodiment of the present invention, access catheter 101 comprises a plurality of slots or grooves that make the catheter body expandable. In one embodiment, expandable element 180 comprises a plurality of slots 181 formed in the catheter body inner wall 108a and/or outer wall 108b. In one embodiment, a pull wire (not shown) can be used to pull a distal region of the catheter proximally, causing the slotted portions to expand, as shown in FIG. 14B. In some embodiments, outer sheath 103 comprises a plurality of slots 181.

### Method of Using Articulating Catheter to Aim and Actively Brace

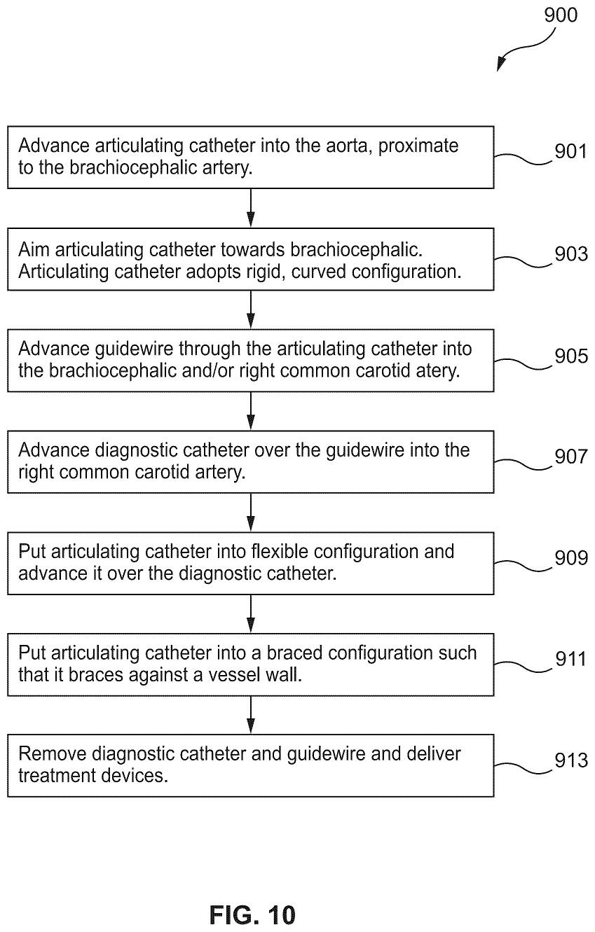

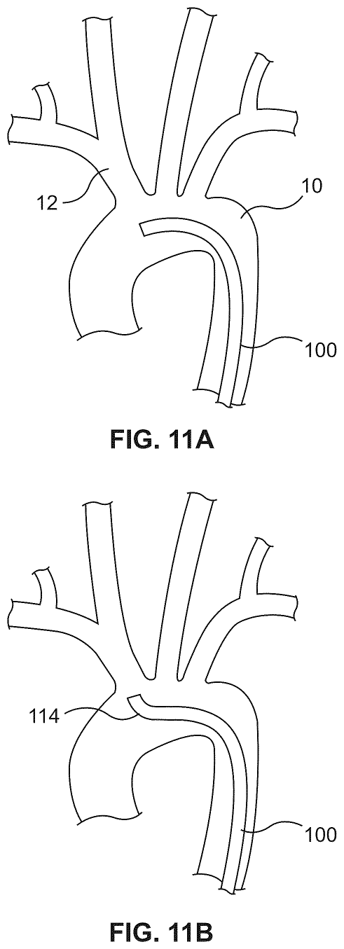

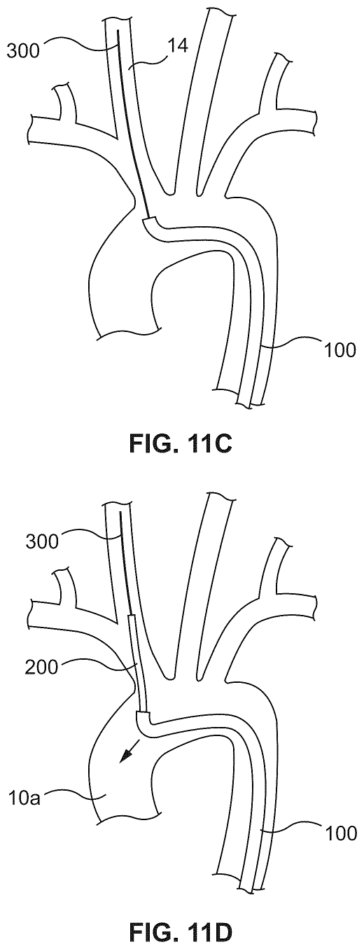

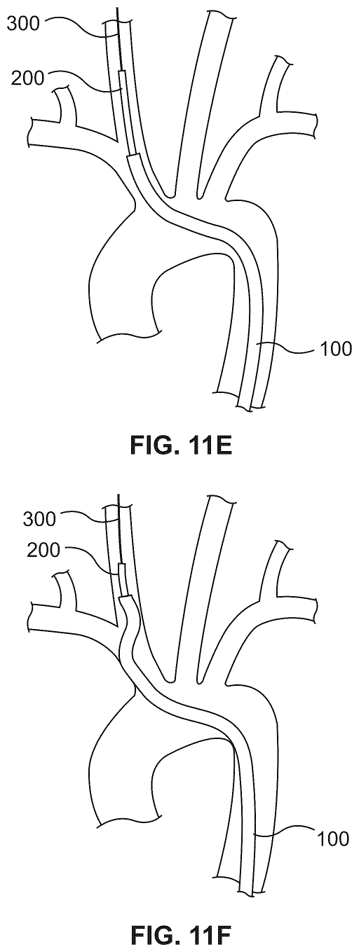

FIG. 10 illustrates a method for advancing devices for treatment of a neurological condition using the catheter access system 1000 disclosed herein. By way of example, the neurological procedure is a mechanical thrombectomy for treatment of acute ischemic stroke, however the method can be used for other procedures described herein. The method may be better understood with reference to FIGS. 11A to 11F. Method 900 uses catheter access system 1000 comprising articulating catheter 100, diagnostic catheter 200, and guidewire 300. In this example, the anatomy has a TYPE III aortic arch, and there is an occlusion (not shown) in a deeper neurovascular vessel, for example, the internal carotid artery, the middle cerebral artery, or another location. The same method may be used to access other vessels as well, for example, the left common carotid artery 18, or the left subclavian artery 20.

Method 900 begins at step 901, where articulating catheter 100 is advanced into the aortic arch 10 to a first location, proximate to the brachiocephalic artery 12. Diagnostic catheter 200 and guidewire 300 may be received within articulating catheter 100, or guidewire 300 may be used to facilitate delivery of articulating catheter 100 to this first location, as shown in FIG. 11A. In some embodiments, articulating catheter 100 tracks over guidewire 300, the latter being inserted into the aortic arch 10 first.

At step 903, a physician aims the articulating catheter 100 by manipulating an actuator 120 on the handle 118, causing the distal region 114 to deflect towards the brachiocephalic artery 12. The articulating catheter 100 adopts a curved and rigid configuration as shown in FIG. 11B. The physician may optionally lock the articulating catheter 100 in this braced configuration. In an embodiment where the double curved articulating catheter 100 is used, the proximal curve may bend around the aortic arch 10 and the distal curve may aim towards the brachiocephalic artery 12, as described herein.

At step 905, guidewire 300 is advanced into the brachiocephalic artery 12 and/or right common carotid artery 14, shown in FIG. 11C.

At step 907, diagnostic catheter 200 is advanced over the guidewire 300 into the brachiocephalic artery 12 and right common carotid artery 14, shown in FIG. 11D. As it is advanced, diagnostic catheter 200 exerts a downward force (i.e., a force biasing the articulating catheter 100 towards the ascending aorta 10a), shown by the arrow in FIG. 11D. While in the braced configuration, articulating catheter 100 counters this force and reduces the risk of prolapse.

At step 909, with the diagnostic catheter 200 advanced far enough into the brachiocephalic artery 12 and/or right common carotid artery 14, such that it is able to passively brace against a vessel wall, the physician may manipulate the actuator such that articulating catheter 100 returns to a flexible/maneuverable configuration and advances the articulating catheter 100 further into vasculature, as shown in FIG. 11E. Articulating catheter 100 tracks over diagnostic catheter 200 and/or guidewire 300.

At step 911, with the articulating catheter 100 advanced further into the brachiocephalic artery 12 or right common carotid artery 14, the articulating catheter 100 may again be manipulated to adopt the braced configuration, such that the articulating catheter 100 actively braces against a vessel wall, as shown in FIG. 11F. Articulating catheter may have either a single curve or a double curve/s-shaped curve as described herein. The curved region(s) of articulating catheter 100 make contact with the vessel wall.

At step 913, with articulating catheter 100 actively braced against a vessel wall, diagnostic catheter 200 and guidewire 300 may be removed from the assembly and one or more treatment devices may be inserted to carry out the rest of the procedure.

In some procedures, the process of bracing the articulating catheter 100, advancing inner devices, returning articulating catheter 100 to a maneuverable configuration, advancing articulating catheter 100, and causing the articulating catheter 100 to adopt a braced configuration again, happen multiple times as the devices are gradually advanced through the neurovasculature.

### Method of bracing with an anchoring mechanism

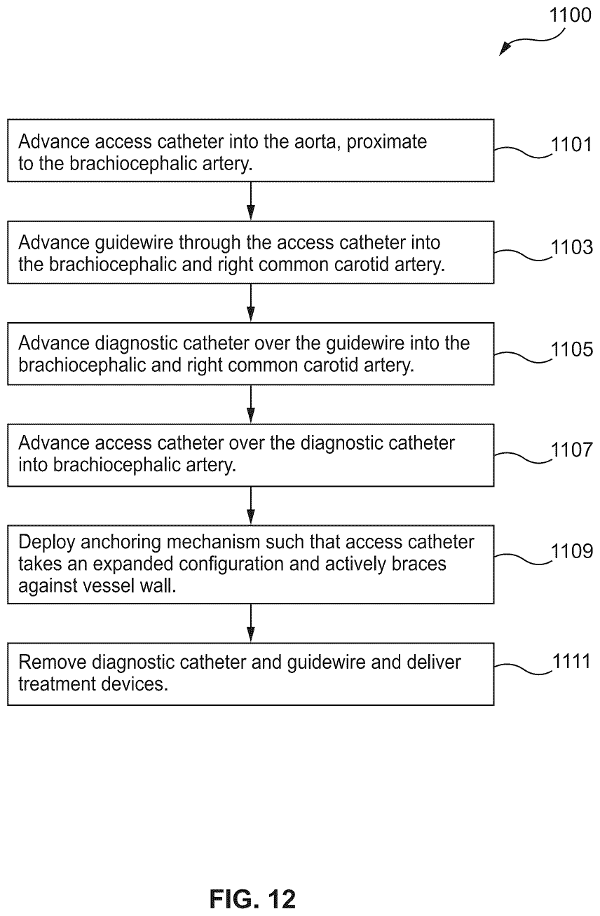

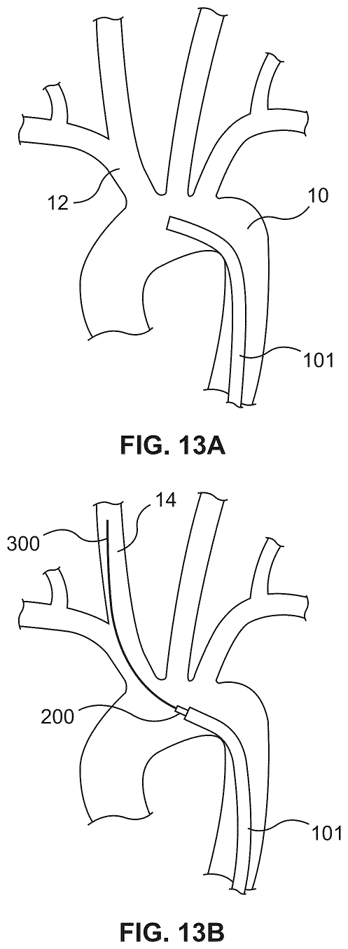

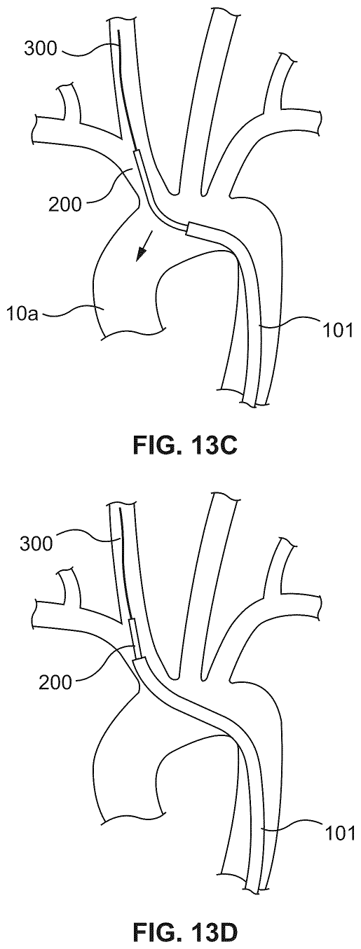

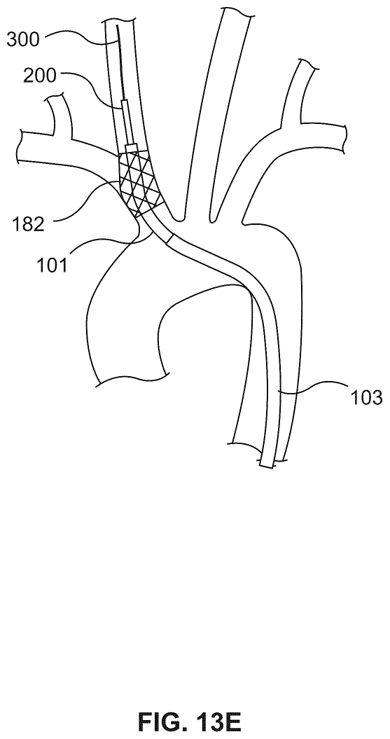

FIG. 12 illustrates a method for advancing devices for a neurological procedure, for example, treatment of acute ischemic stroke, using another embodiment of the catheter access system 1000 disclosed herein. The method may be better understood with reference to FIGS. 13A to 13E. Method 1100 uses catheter access system 1000 comprising access catheter 101, diagnostic catheter 200, and guidewire 300. Access catheter 101 comprises an anchoring mechanism described herein Similar to the example of method 900, the anatomy has a TYPE III aortic arch.

Method 1100 begins at step 1101, where access catheter 101 is advanced into the aortic arch 10 to a first location, proximate to the brachiocephalic artery 12. Diagnostic catheter 200 and guidewire 300 may be received within access catheter 101, or guidewire 300 may be used to facilitate delivery of access catheter 101 to this first location, as shown in FIG. 13A.

At step 1103, guidewire 300 is advanced into the brachiocephalic artery 12 and right common carotid artery 14, shown in FIG. 13B.

At step 1105, diagnostic catheter 200 is advanced over the guidewire into the brachiocephalic artery 12, shown in FIG. 13C. As it is advanced, diagnostic catheter 200 may exert a downward force, represented by the arrow, i.e., a force biasing access catheter 101 and/or diagnostic catheter 200 towards the ascending aorta 10a.

At step 1107, with the diagnostic catheter 200 advanced partially into the brachiocephalic artery 12, access catheter 101 is advanced such that a distal region is in the brachiocephalic artery 12, shown in FIG. 13D.

At step 1109, the anchoring mechanism is deployed, such that it takes an expanded configuration and actively braces against the vessel wall. FIG. 13E shows access catheter 101 comprising a scaffold 182 described herein. In other embodiments, access catheter 101 comprises any other anchoring mechanism described herein, for example a non-occlusive balloon assembly 152. In some embodiments, catheter access system 1000 comprises outer sheath 103, which may be advanced with access catheter 101. (NOTE: outer sheath 103 is not shown in FIGS. 13A to 13D)

At step 1111, with access catheter 101 actively braced against a vessel wall, diagnostic catheter 200 and guidewire 300 may be removed from the assembly and treatment devices may be inserted to carry out the rest of the procedure.

As an optional step 1110, between 1109 and 1111, guidewire 300 and diagnostic catheter 200 may be advanced further into the neurovascular system where they may passively brace, anchoring mechanism may be contracted and access catheter 101 is advanced further into the vessel, then anchoring mechanism may be expanded again, this time deeper in the vasculature, for example in the right common carotid artery 14. In other words, the process of bracing the outer device, advancing inner devices, returning to a maneuverable configuration, advancing outer device, bracing outer device, may happen multiple times as the devices are gradually advanced through the neurovasculature.

In some embodiments, for either method 900 or method 1100, diagnostic catheter 200 may not be used and articulating catheter 100/access catheter 101 may be used with guidewire 300 until articulating catheter 100/access catheter 101 is braced at a desired location.

The systems and methods disclosed herein can be used for other applications that require delivering devices to the neurovascular system, including mechanical thrombectomy, stenting, coiling, pipeline/flow diversion, stent-assisted coiling, aneurysm embolization, and balloon assisted coiling. Another application may be for lead placement in the heart where stabilizing a lead in a complex cardiac environment is difficult.

The embodiments of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation, or identification of any reference in this application shall not be construed as an admission that such reference is available as prior art to the present invention.

1. A method for positioning a device within a target blood vessel, the method using a medical device assembly including an inner device and an outer device, the outer device configured to have a maneuverable configuration for advancing through vasculature, and a braced configuration for supporting advancement of the inner device therethrough, the method comprising the steps of:
advancing the outer device to a first location within a patient's body while the outer device is in the maneuverable configuration;
causing the outer device to adopt the braced configuration at the first location; and
advancing the inner device through the outer device into the target blood vessel while the outer device is maintained in the braced configuration;
wherein, while in the braced configuration, the outer device is configured to substantially maintain its position at the first location to facilitate advancement of the inner device therethrough to the target blood vessel.

2. The method of claim 1, wherein while in the braced configuration, the outer device comprises a curved orientation.

3. The method of claim 2, wherein the curved orientation comprises an S-shaped curve.

4. The method of claim 2, wherein the first location is within the target blood vessel, and wherein a curved portion of the outer device is in contact with a wall of the target blood vessel while in the braced configuration.

5. The method of claim 3, wherein the first location is within the target blood vessel, and wherein a first curved portion of the outer device and a second curved portion of the outer device are in contact with a wall of the target blood vessel while in the braced configuration.

6. The method of claim 1, wherein the step of causing the outer device to adopt the braced configuration comprises deploying an anchoring mechanism.

7. The method of claim 6, wherein the anchoring mechanism comprises a non-occlusive balloon assembly.

8. (canceled)

9. The method of claim 6, wherein the anchoring mechanism comprises an expandable element.

10. (canceled)

11. The method of claim 1 further comprising a step of locking the outer device in the braced configuration.

12. The method of claim 11, wherein the step of locking the outer device comprises manipulating a locking mechanism.

13. The method of claim 1, further comprising a step of, following the step of advancing the inner device, causing the outer device to adopt the maneuverable configuration and advancing the outer device to a second location, wherein the second location is closer to the target blood vessel than the first location.

14. The method of claim 1, further comprising a step of, following the step of advancing the inner device, causing the outer device to adopt the maneuverable configuration and advancing the outer device into the target blood vessel.

15. The method of claim 1, wherein the target blood vessel is selected from the group consisting of: a right common carotid artery, a left common carotid artery, an internal common carotid artery, a left subclavian artery, a right subclavian artery, and a brachiocephalic artery.

16. The method of claim 1, wherein advancing the outer device to the first location includes advancing the outer device within an aorta.

17. (canceled)

18. The method of claim 1, further comprising a step of advancing at least one treatment device through the outer device.

19. The method of claim 18 further comprising a step of treating a neurological condition using the at least one treatment device.

20.-23. (Cancelled)

24. The method of claim 2, wherein the step of causing the outer device to adopt a braced configuration comprises manipulating an actuator.

25. The method of claim 3, wherein the step of causing the outer device to adopt a braced configuration comprises manipulating an actuator, wherein the actuator is coupled to a first location and a second location on the outer device.

26. The method of claim 3, wherein the step of causing the outer device to adopt a braced configuration comprises manipulating a first actuator and a second actuator, the first actuator coupled to a first location on the outer device and the second actuator coupled to a second location on the outer device.

27. The method of claim 1, wherein causing the outer device to adopt the braced configuration includes aiming a distal end of the outer device towards the target blood vessel.
